(19) United States
(12) Patent Application Publication
(10) Pub. No.: US 2005/0177136 A1
(43) Pub. Date: Aug. 11, 2005

(54) EXTERNALLY DISPOSED PUMP FOR USE WITH AN INTERNALLY MOUNTED AND COMPLIANT CATHETER

(76) Inventor: Landon C.G. Miller, Tuscaloosa, AL (US)

Correspondence Address: GIFFORD, KRASS, GROH, SPRINKLE & CITKOWSKI, P.C, PO BOX 7021, TROY, MI, 48007-7021, US

(21) Appl. No.: 11/017,297

(22) Filed: Dec. 20, 2004

Related U.S. Application Data
(60) Provisional application No. 60/531,487, filed on Dec. 19, 2003.

(51) Int. Cl. A61K 9/22

(52) U.S. Cl. 604/890.1

(57) ABSTRACT

A therapeutic agent delivery pump includes a bottle having a pressurant chamber containing a pressurant and a therapeutic agent volume, the volume terminating in a visco-elastic septum. The bottle has a piercing element in alignment with the septum and in fluid communication with tubing. A flow restriction gauge impinges on the tubing to restrict the flow of a therapeutic agent from the balloon. An intrathecal drug delivery system includes a pump as described above, along with a pressure compatible catheter. A connector is provided between the pump and the catheter. An externally disposed therapeutic pump assembly for administering a therapeutic delivery agent in metered fashion to an internal location of a patient includes a three-dimensional body, with a lid secured to the body. A bag holding a volume of therapeutic delivery agent contained within the body in order to progressively compress.

### RELATED APPLICATION

This application claims priority of U.S. Provisional Patent Application Ser. No. 60/531,487 filed Dec. 19, 2003, which is incorporated herein by reference.

### FIELD OF THE INVENTION

The present invention generally relates to an external therapeutic agent delivery pump intended for coupling to a catheter. In particular, the present invention teaches an external pump that urges a therapeutic agent from the pump, such as under a hydrostatic pressure, for delivery to an internally mounted and pump compliant catheter.

### BACKGROUND OF THE INVENTION

Catheter implantation has become a standard medical procedure in order to treat chronic cerebrospinal conditions such as pain. Subsequent to initial catheter implantation, a sophisticated therapeutic agent delivery pump is required in order to optimize therapeutic agent delivery dosimetry. However, with control of intrathecal dosimetry, devoting a complex pump for perpetual therapeutic administration becomes a considerable portion of the cost associated with intrathecal drug delivery. Thus, there exists a need for a simple, disposable external pump for intrathecal administration.

### SUMMARY OF THE INVENTION

A therapeutic agent delivery pump includes a bottle having a pressurant chamber containing a pressurant and a therapeutic agent volume, the volume terminating in a visco-elastic septum. The bottle has a piercing element in alignment with the septum and in fluid communication with tubing. A flow restriction gauge impinges on the tubing to restrict the flow of a therapeutic agent from the balloon.

An intrathecal drug delivery system includes a pump as described above, along with a pressure compatible catheter. A connector is provided between the pump and the catheter.

An externally disposed therapeutic pump assembly for administering a therapeutic delivery agent in metered fashion to an internal location of a patient includes a three-dimensional body, with a lid secured to the body. A bag holding a volume of therapeutic delivery agent contained within the body in order to progressively compress and administer the therapeutic agent in metered fashion to a catheter tube extending from the body. The catheter tube is in communication with the internal delivery location of the patient.

### BRIEF DESCRIPTION OF THE DRAWINGS

Reference will be made to the attached drawings, when read in combination with the following detailed description, wherein like reference numerals refer to like parts throughout the several views, and in which:

FIG. 1 is a schematic showing an inventive dispensing pump as part of an intrathecal therapeutic agent delivery system;

FIG. 2 is a partial cutaway view of an inventive pump;

FIG. 3 is an exploded view of a step-down hydrostatic pressure regulator component of an inventive pump;

FIG. 4 is an expanded view of a mechanical caliper component of an inventive pump for metering therapeutic agent;

FIG. 5 is a partial cutaway and exploded view of a therapeutic agent bottle according to the present invention;

FIG. 6 is an alternate embodiment of a bottle according to the present invention depicting alternate port locations for pressurizing gas administration into a bottle;

FIG. 7 is another embodiment of a bottle according to the present invention depicting alternate port locations for spring or gas-driven cylinder administration into a bottle;

FIG. 8 is a perspective view of a rack and pinion pump assembly according to a further preferred embodiment of the present invention;

FIG. 9 is an exploded view of the pump assembly of FIG. 8 and further illustrating the components of the track, roller and progressively compressible therapeutic agent containing bag;

FIG. 10 is an enlarged sectional view of the interengaging relationship between the cylinder track bed and cylinder bag roller;

FIG. 11 is an exploded view of a flat spring variant of therapeutic agent delivery pump according to a further preferred embodiment of the present invention;

FIG. 12 is an exploded view of the compressible bag and regulating delivery components associated with the embodiment of FIG. 11; and

FIG. 13 is an exploded view of a therapeutic delivery pump assembly according to a further preferred embodiment of the present invention and which includes an internally pressurized fill bottle in combination with an extending catheter tubing and associated regulator valve.

### DETAILED DESCRIPTION OF THE PREFERRED EMBODIMENTS

The present invention has utility as a pump for intrathecal therapeutic agent delivery. By removing complex mechanical and electronic components from an inventive pump, intrathecal drug delivery is achieved with greater efficiency than previously performed. While the inventive pump is contemplated to be disposable, it is appreciated that pump reloading and recycling are readily performed.

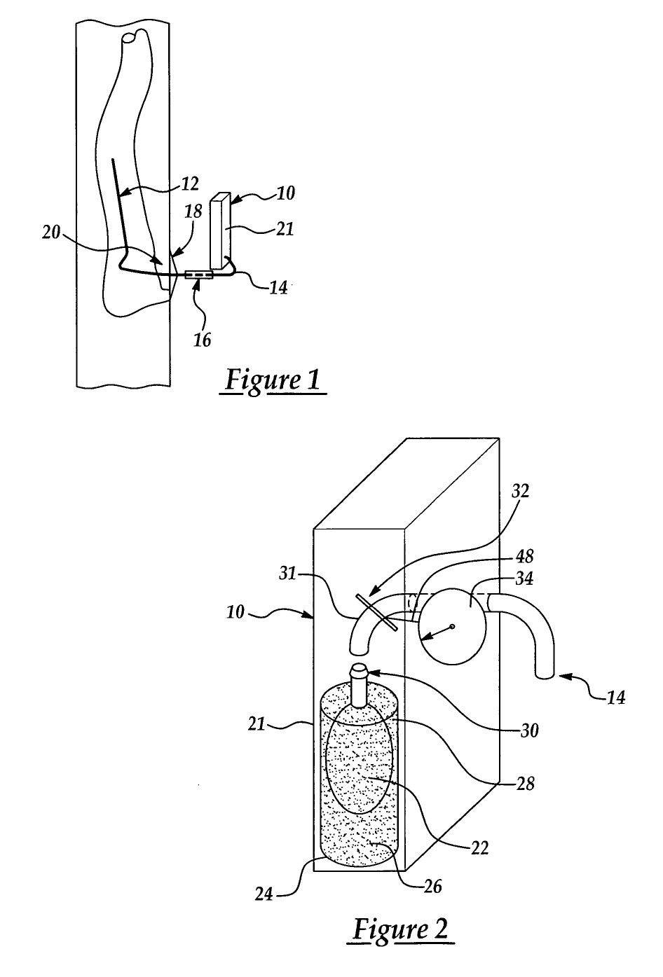

Referring now to FIGS. 1-3, an inventive external pump is shown generally at 10 as part of an intrathecal therapeutic agent delivery system. The pump 10 is coupled to an internal pump compliant catheter 12 by way of conventional pump compliant polymeric tubing 14.

An inline antimicrobial filter 16 (such as for example exhibiting a 0.22 micron rating) is preferably situated intermediate between the tubing 14 and catheter 12. As is conventional with catheter implantation, the skin entrance point for the catheter is overlaid with an antimicrobial (anti-bacterial, anti-viral) shield 18. Additionally, underlying the shield 18 is a catheter stabilization mesh 20 functioning to retain catheter position and orientation. It is appreciated that while the inventive pump 10 is detailed with respect to a typical implanted catheter, other variants of this catheter are operative herein.

With particular reference to FIG. 2, an inventive pump 10 includes a housing 21 and a therapeutic agent balloon 22. As used herein, a “therapeutic agent” is defined to include any drug or compound administered to promote a nutritional, therapeutic or other desired effect on a biological subject, the agent having a nutrient, stimulating action, inhibition, analgesic, destructive or regulatory effect on the subject where a subject is recognized to include a human, primate, domesticated animal and a plant.

The therapeutic agent balloon 22 is encompassed within a pressurized bottle 24. The surrounding pressurant illustratively includes a halocarbon, dinitrogen, a noble gas, carbon dioxide in combination thereof. The pressurant located within a pressurant chamber 26 exerts an external pressure on the drug balloon 22 that is compressible in response to the pressure exerted by the pressurant 26.

A pierceable cover 28 overlies the drug balloon 22 in a static condition prior to desired administration. Preferably, the cover 28 includes an antimicrobial coating operative to sterilize a piercing element 30. The piercing element 30 penetrates the cover and enters the therapeutic agent balloon 22 in order to provide a route for therapeutic agent to escape from the bottle 24. Preferably, the piercing element 30 is in fluid communication with a pump tubing 31.

Intermediate between piercing element 30 and catheter tubing 14, a mechanical caliper 32 restricts the flow of therapeutic agent from said balloon 22. A gauge 34 affords metering control of therapeutic agent from the balloon 22 into catheter tubing 16. Preferably, the gauge 34 is accessible on the exterior of a pump housing 36. More preferably, the gauge 34 is recessed in the housing 36 such that upon adjustment to a predetermined position, the gauge 34 is protected against inadvertent jostling.

A therapeutic agent balloon 22 according to the present invention is formed of a drug compatible visco-elastic material molded to a thickness compatible with storage under pressure conditions associated within the bottle 24. The bottle 24 is formed with a material capable to withstand the internal pressure therein of generally less than 100 atmospheres. Preferably, the pressurant is stored at a pressure between 3 and 20 atmospheres. The bottle 24 is illustratively formed of steel, aluminum, and high-impact plastic materials.

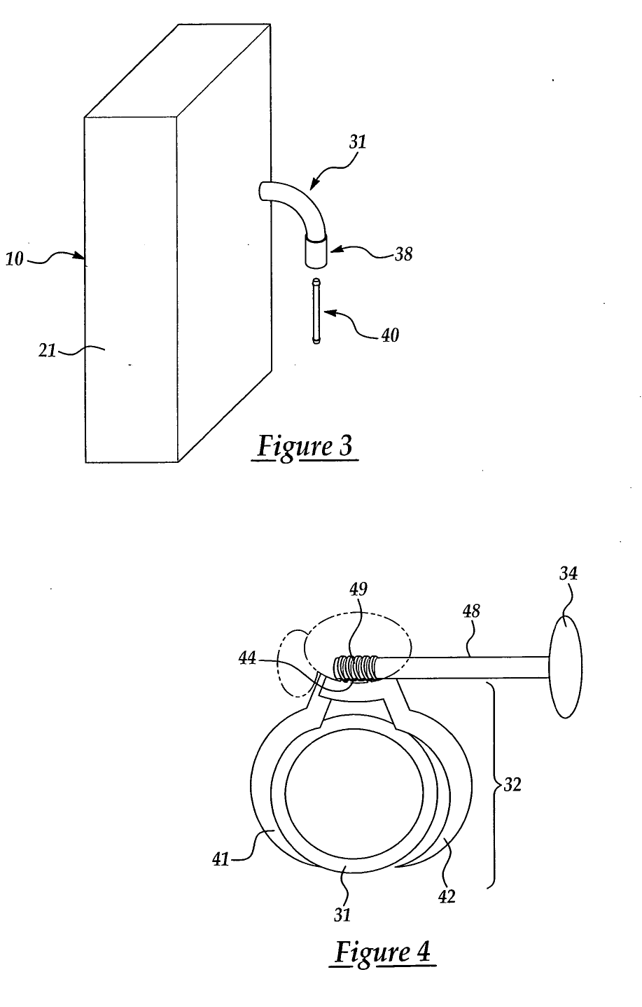

With particular reference to FIG. 3, an inventive pump 10 is coupled to tubing 14 by way of a conventional connector 38. Preferably, the connector 38 includes backward slanting external edges to inhibit inadvertent decoupling. While the coupler 38 is depicted as a linear connection between the pump tubing 31 and tubing 14, it is appreciated that Y- and T-shaped connectors are illustratively operative herein in instances where one wishes to couple additional accessories in the tubing 14.

Representative accessories illustratively include a pressure transducer, a drug delivery port, and a second therapeutic agent delivery pump of conventional or inventive design. Preferably, intermediate between inventive pump tubing 31 and catheter 12, a hydrostatic pressure interface plug 40 is provided that reduces the tubing diameter in fluid communication with the therapeutic agent to a smaller cross-sectional area relative to the tubing 14.

It is appreciated that a plug 40 is alternatively placed in the pump tubing 31, or in the tubing connector 38. Typically, the plug reduces the tubing internal cross-sectional area between 20 and 90 percent and thereby affords a pressure magnifying effect within the tubing 16 and catheter 12 so as to offer greater control over therapeutic agent administration.

Referring now to FIG. 4, the caliper 32 is shown in an operational relationship relative to the gauge 34 and pump tubing 31. The caliper 32 includes two opposing arms 41 and 42. Preferably, the arms 41 and 42 each have an internal contoured surface generally complementary to the external surface of pump tubing 31. Preferably, arm 41 is stationary while arm 42 is hingably moveable relative to arm 41 through enmeshment of worm gear teeth 44 with complementary threads 46 associated with a gauge shaft 48, the gauge shaft 48 terminating in the gauge 34. Thus, dial settings are provided on the gauge 34 as a percentage of total flow rate. For example, baclofen is provided at a 100% flow rate of 2500 micromiliters per day. The gauge gears 49 are preferably calibrated such that each increment of rotation corresponds to a predetermined percentage of total flow. Typical flow ranges are between 10% and 100% with increments of from 1% to 10% total maximum flow rate. Preferably, the increment is 5%.

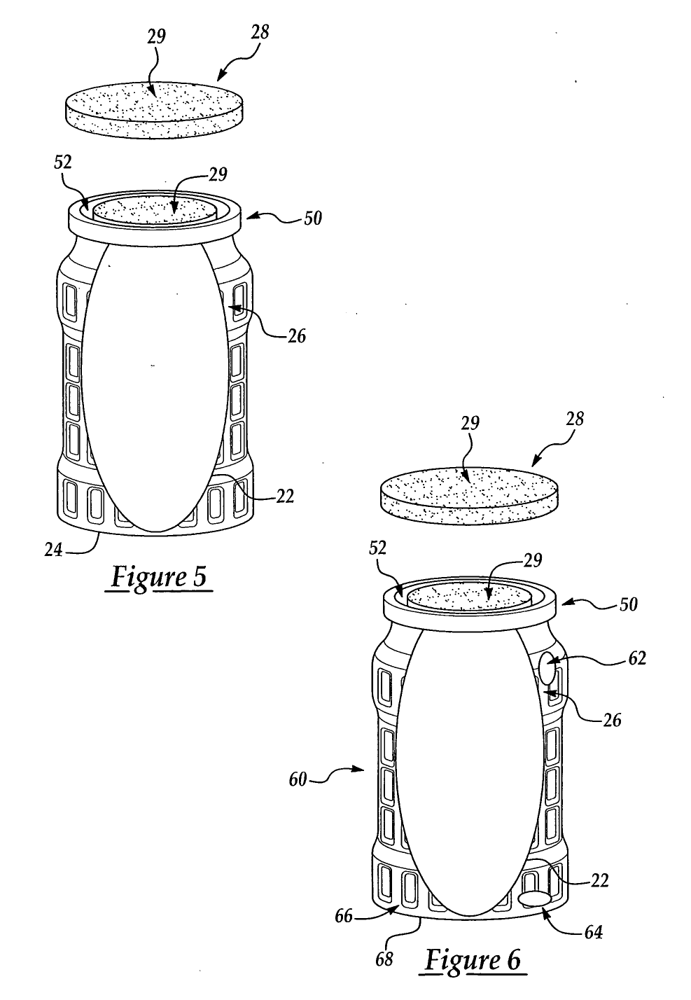

Referring now to FIG. 5, an inventive bottle 24 is shown in greater detail. The bottle 24 defines a pressurant chamber 26 in which the therapeutic balloon 22 is secured. Access to the therapeutic agent balloon 22 is by way of a visco-elastic septum 50. The septum 50 optionally includes an inlet 52 in fluid communication with the pressurant chamber 26 so as to facilitate separate filling thereof. Preferably, the septum 50 has an antimicrobial coating 29 in order to inhibit microbial contamination of the therapeutic agent.

The antimicrobial coating 29 illustratively includes a dried layer of topical antiseptics illustratively including chlorhexadine, Betadine, undecylenic acid, and/or benzalkonium chloride. The antimicrobial coating 29 is further protected by the pierceable cover 28. The cover 28 formed of materials illustratively including metal foils, and plastics. Preferably, the cover 28 creates a sterile volume between the intersurface 55 thereof and the septum 50. More preferably, the top surface 56 of the cover 28 also has an antimicrobial coating 29.

In an alternate embodiment of the inventive bottle depicted in FIG. 6, the bottle is shown generally at 60 where like numerals correspond to the descriptions with respect to FIG. 5. The bottle 60 in contrast to bottle 22 affords access to the pressurant chamber through an inlet 62 located in the wall 64 of the bottle 60. Alternatively, an inlet 66 is provided in the base 68 of the bottle 60. An inlet port such as 62 or 64 in concert with an aligned aperture in an external pump housing 21 affords one the ability to monitor and recharge the pressurant chamber 26 during the course of storage.

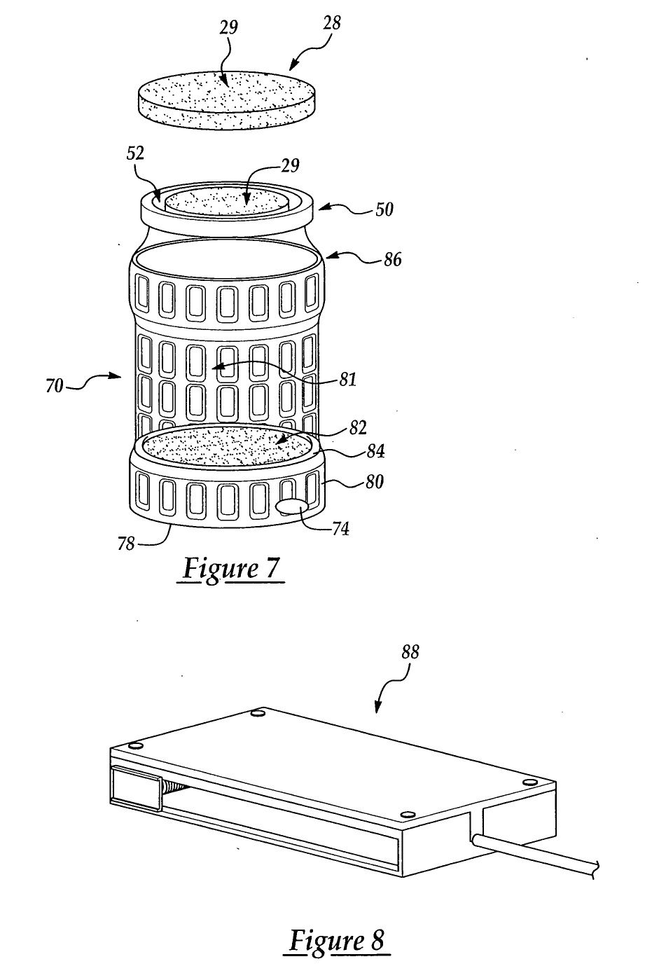

In another embodiment of the inventive bottle depicted in FIG. 7, the bottle is shown generally at 70 where like numerals correspond to the descriptions with respect to FIG. 5. The bottle 70 in contrast to bottle 22 affords access to the pressurant chamber through an inlet 76 provided in the base 78 of the bottle 70. A mechanical spring 80 and/or a gas driven cylinder 82 urges a therapeutic agent from the balloon 22, or as depicted in FIG. 7 a therapeutic agent space 81 above the cylinder 82. The cylinder 80 has an O-ring seal 84 with the interior wall of the bottle 70. A cylinder stop-notch ring 86 is provided on the interior wall of the bottle 70 to preclude damage to a piercing element withdrawing therapeutic agent from a balloon within the bottle 70.

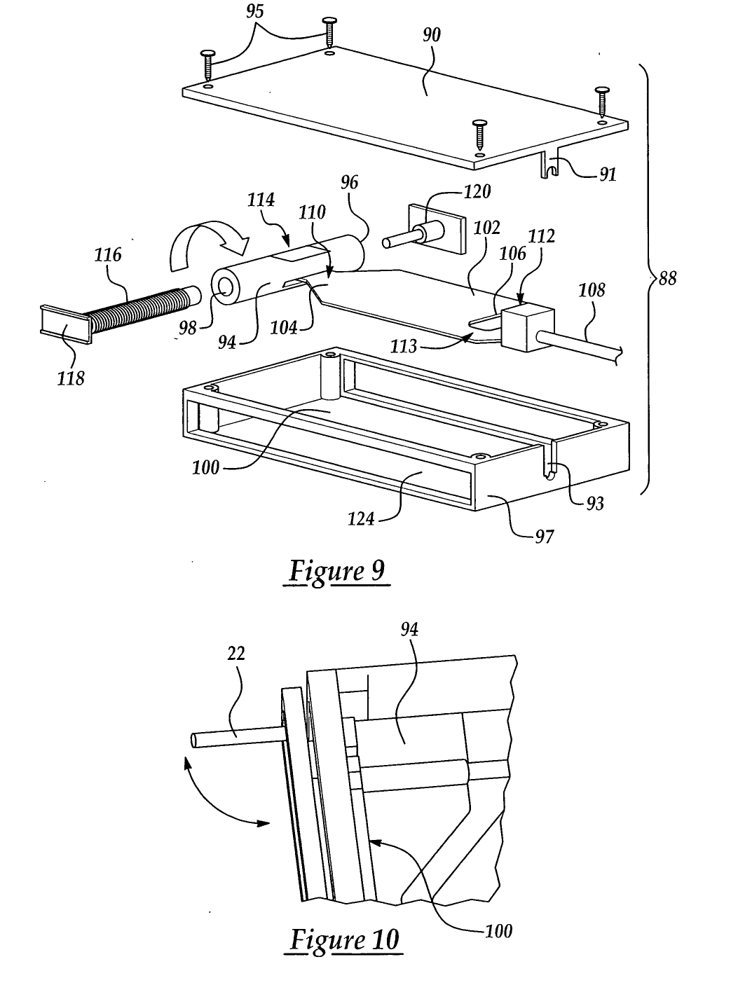

Referring now to FIGS. 8 and 9, both perspective and exploded views are shown of a rack and pinion pump assembly 88, according to a further preferred embodiment of the present invention. The rack and pinion assembly provides an alternate arrangement for establishing a measured release of a therapeutic agent and includes a lid 90 secured over an interiorly hollowed (typically rectangular and box-shaped) base 92. A downwardly extending latch 91 associated with the lid 90 seats within an aperture 93 associated with an end of the base 92 in order to lock in place a catheter tube (as subsequently referenced at 108). Additionally, screws 95 are provided to secure the lid 90 to the base such that the screws pass through aligning and mating apertures as illustrated.

Seated within the base 92 is a geared roller 94, this including circumferentially geared ends 96 and 98 which each seat upon a cylinder track bed, see as shown along one extending side at 100, in both FIGS. 9 and 10. A compressible, sterilized bag 102 contains a desired fluidic agent and includes a first end 104 secured to a location of the geared roller 94 and such that, upon progressive rotation and translation of the roller 94, the bag 102 is progressively wound about the outer circumferential area of the roller and its internal contents are progressively squeezed through an opposite end 106 located at a terminal point of the assembly and fluidly communicable with a catheter connecting tube 108.

A mixture injection point 110 is associated with the first end 104 of the compressible bag. In use, a control valve 112 is located at the end of the bag 102 proximate the catheter tube 108 and/or establishing a measured outflow of agent (such as rates including 1, 0.75 or 0.50 ml per diem) and which is set before the bag is filled and inserted within the base enclosure. Although not clearly shown, a needle 113 is associated with the second end 106 to facilitate outflow to the control valve 112 and catheter tube 108.

A tape backing is associated with the first end 104 of the bag which his slipped under and over the roller 94. A tape backing 114 associated with the geared roller is removed and, upon winding of the roller, results in the bag being progressively wrapped thereabout. A counter-wound coil spring 116 is secured about an axle 118 and seats against a first end of the gear roller 94. An opposing support axle 120 seats an opposite end of the gear roller 94 (again in proximity to the geared ends 96 and 98) and, upon the winding action of a crank (see at 122 in FIG. 10), a sufficient degree of compression is maintained on the bag 102 in order to maintain sufficient pressure to empty the bag in an even and metered fashion. Additional features include an elongated slot 124 being formed along an extending side of the base 92 to provide for visual inspection of the volume of fluid remaining in the bag 102.

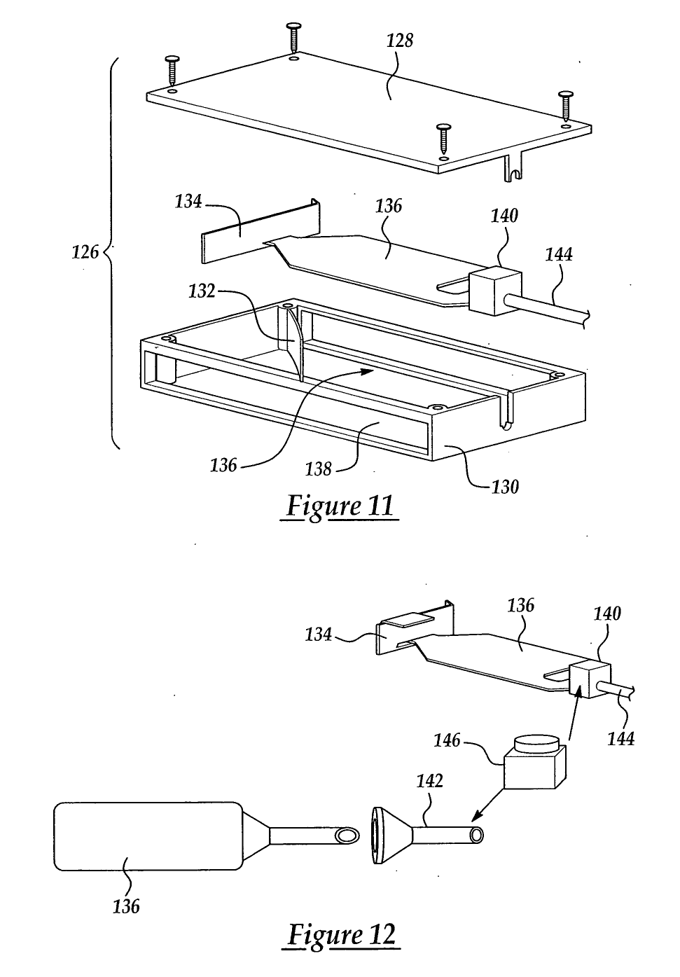

FIG. 11 is an exploded view is shown at 126 of a flat spring variant of therapeutic agent delivery pump according to a further preferred embodiment of the present invention. A lid 128 and base 130 are provided, similar to the arrangement illustrated in the embodiment of FIGS. 8-10.

The base 130 includes an alternate configuration in the form of an angled and inwardly extending flat spring 132, the purpose for which is to apply a consistent degree of pressure to a widthwise extending plate 134, in turn secured to a fluid filled and compressible bag 136. The plate 134 is mounted such that it translates along internal and smooth guides, see at 136 and 138, to thereby maintain the necessary compressive force to empty the bag in metered fashion and with the optional assistance of a regulator component 140 arranged intermediate a welded-on plastic needle 142 (see FIG. 12) and a catheter needle 144.

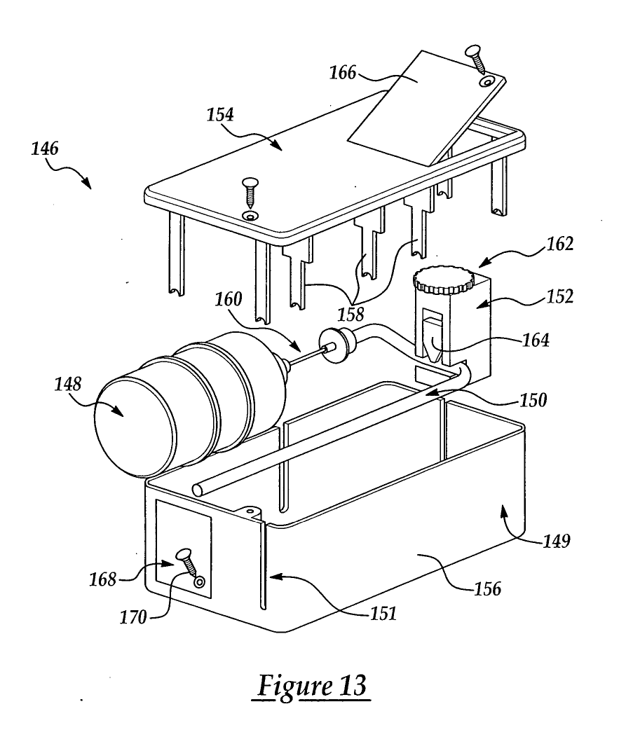

FIG. 13 is an exploded view, at 146, of a therapeutic delivery pump assembly according to a further preferred embodiment of the present invention and which includes an internally (gas) pressurized fill bottle 148 in combination with an internal to external extending catheter tubing 150 and associated (screw down) regulator valve 152. A lid 154 is secured atop an interiorly open base 156, the lid including provision of locator setoffs 158, the purpose for which being to secure the internal catheter tubing 150 in place during assembly.

As previously described, the bottle 148 is internally pressurized such that, upon being pierced by a catheter bottle needle 160, a fluid therapeutic delivery agent is expelled into the catheter tubing 150. The regulator valve 152 includes a rotatable dial 162, the purpose for which being to adjust the degree of pinching (compression) of a downwardly displaceable component 164, in turn affecting the volume of fluid administered in metered fashion through the tubing 150.

A door 166 is pivotally mounted in the lid 154 and is opened to facilitate regulator adjustment. An alternative arrangement includes a pump body cover 168 formed in the base 156 and operable with a hinge and screw arrangement 170 to open and close a portion of the body to reveal the end of the internally pressurized bottle 148, and such as to permit replacement thereof. Combination drainage holes 149 and a catheter tubing opening 151 are provided at locations along the body 156 as shown.

It is appreciated that an inventive pump is readily designed to be amenable to the insertion of bottles containing different sizes of therapeutic agent balloons. Preferably, different size balloons are accommodated by modifying the bottle area without significantly changing the relative position of the septum relative to pump components.

The process of charging a bottle with a therapeutic agent according to the present invention includes bottle sterilization. The balloon and septum are likewise sterilized in a seal formed between the septum and the pressurant chamber. Such a seal is readily formed, the methods conventional to the art illustratively including a threaded securement, sonic welding, and chemical adhesives. Upon testing the pressurant chamber seal and the separation thereof from therapeutic agent balloon contents, a pressurant is injected into the pressurant chamber either through a septum edge inlet as depicted in FIG. 5, or through an inlet 62 or 66 or 76, as depicted in FIGS. 6 and 7.

Thereafter, an antimicrobial coating is applied to the septum and a therapeutic agent injected through the septum into the therapeutic agent balloon. Cover application and the application of an antimicrobial coating complete the bottle assembly process. Upon piercing the cover and septum, the therapeutic agent is allowed to flow past the restrictions created by a mechanical caliper and an optional hydrostatic plug so as to administer the therapeutic agent at a preselected rate to administration apparatus in contact with a subject.

It is appreciated that one skilled in the art upon reading the above description will recognize various modifications to the invention described herein that do not depart from the spirit of the invention. These modifications are intended to be encompassed by the appended claims.

1. A therapeutic agent delivery pump comprising:
a bottle having a pressurant chamber containing a pressurant and a therapeutic agent volume, the volume terminating in a visco-elastic septum;
a piercing element in alignment with the septum and in fluid communication with tubing; and
a flow restriction gauge impinging on the tubing to restrict the flow of a therapeutic agent from the balloon.

2. The pump of claim 1 further comprising a pressurant chamber inlet in a wall of said bottle.

3. The pump of claim 2 wherein the wall is a bottom wall.

4. The pump of claim 1 wherein said restrictor is a mechanical caliper.

5. The pump of claim 1 further comprising a tubing cross-sectional area reducing plug.

6. The pump of claim 1 wherein said restrictor gauge is accessible from the exterior of said pump housing.

7. The pump of claim 6 wherein said gauge is recessed into the pump housing.

8. The pump of claim 1 wherein the pressurant is selected from the group consisting of: halocarbons, dinitrogen, noble gases, and carbon dioxide.

9. The pump of claim 1 further comprising a cover pierceable by said piercing element and intermediate between the piercing element and the septum.

10. The pump of claim 1 further comprising an antimicrobial coating on the septum.

11. An intrathecal drug delivery system comprising:
a pump according to claim 1;
a pressure compatible catheter; and
a connector therebetween.

12. The system of claim 11 further comprising an antimicrobial filter between said pump and said catheter.

13. The system of claim 11 wherein said connector further couples an accessory to said pump and said catheter.

14. The system of claim 13 wherein said accessory is selective from the group consisting of: a pressure transducer, a therapeutic agent injection portal, and a drug delivery pump.

15. The system of claim 11 wherein said catheter is implanted within a subject.

16. The system of claim 1 wherein said therapeutic agent volume is defined by a balloon.

17. An externally disposed therapeutic pump assembly for administering a therapeutic delivery agent in metered fashion to an internal location of a patient, said assembly comprising:
a three-dimensional body;
a lid secured to said body; and
a bag holding a volume of therapeutic delivery agent contained within said body, said bag being progressively compressed in order to administer said therapeutic agent in metered fashion to a catheter tube extending from said body and in communication with the internal delivery location of the patient.

18. The pump assembly as described in claim 17, further comprising a gear roller traversable along a track bed within said body, an end of said bag being secured to said roller and such that said bag is compressed and progressively wound about said bag.

19. The pump assembly as described in claim 17, further comprising a crank assembly for actuating said gear roller, a helical spring providing a biasing contact between said spring and said compressible bag.

20. The pump assembly as described in claim 17, further comprising a regulator valve in communication with an outlet of said bag, a catheter connector tube extending from said valve.

21. The pump assembly as described in claim 17, further comprising a flat spring progressively traversable along said base and acting upon a crosswise extending plate in turn secured to said bag and such that said bag is compressed.

22. An externally disposed therapeutic pump assembly for administering a therapeutic delivery agent in metered fashion to an internal location of a patient, said assembly comprising:
a three-dimensional body;
a lid secured to said body; and
an internally pressurized and fluid holding vessel contained within said body, said vessel administering a therapeutic agent in metered fashion to a catheter tube extending from said vessel and in communication with the internal delivery location of the patient.

23. The pump assembly as described in claim 22, further comprising a screw down regulator valve in communication with an outlet location of said catheter tube.

24. The pump assembly as described in claim 22, further comprising a piercing bottle needle extending from an inlet end of said catheter tube and inserted within said internally pressurized vessel.
